(19) United States
(12) Patent Application Publication
(10) Pub. No.: US 2021/0137392 A1
(43) Pub. Date: May 13, 2021

(54) ELECTROCARDIOGRAM MEASUREMENT METHOD AND SYSTEM USING WEARABLE DEVICE

(71) Applicant: WELLBEINGSOFT INC., Daejeon (KR)

(72) Inventor: In-Duk HWANG, Sejong (KR)

(73) Assignee: WELLBEINGSOFT INC., Daejeon (KR)

(21) Appl. No.: 17/256,570

(22) PCT Filed: Jun. 28, 2019
(86) PCT No.: PCT/KR2019/007918

§ 371 Date: Dec. 28, 2020

(30) Foreign Application Priority Data
Jun. 28, 2018 (KR) 10-2018-0074824

(51) Int. Cl. A61B 5/0205; A61B 5/361; A61B 5/332; A61B 5/282; A61B 5/00; A61B 5/271

(52) U.S. Cl. CPC A61B 5/0205; A61B 5/361; A61B 5/332; A61B 5/282; A61B 2560/04; A61B 5/271; A61B 5/7225; A61B 5/681; A61B 5/746; A61B 5/0006

(57) ABSTRACT

The present invention relates to an electrocardiogram measurement system using a wearable device, comprising a photoplethysmograph, and an electrocardiograph provided in a wearable device or an electrocardiograph which can be separated from the wearable device and carried, wherein the photoplethysmograph comprises a photoplethysmogram measurement circuit comprising an LED and a photodiode, an AD converter connected to an output terminal of the photoplethysmogram measurement circuit, for converting an analog signal to a digital signal, a wireless communication means for transmitting and receiving data, and a microcontroller for measuring photoplethysmogram, the microcontroller extracts photoplethysmogram parameters by analyzing the measured photoplethysmogram, determines generation of an alarm by using the extracted photoplethysmogram parameters, and generates an alarm on the basis of the determination result, and the electrocardiograph comprises three dry electrocardiogram measurement electrodes and two amplifiers for amplifying two electrocardiogram signals induced at two electrocardiogram electrodes out of the three electrocardiogram electrodes.

### TECHNICAL FIELD

The present invention relates to an electrocardiogram measurement method and a system using a wearable device, and more particularly, to a method and a system that continuously analyzes a heart rate (HR), a heart rate variability (HRV) and a breathing rate (BR) using one photoplethysmograph and generates an alarm to a user upon detection of an arrhythmia symptom, such that the user measures an electrocardiogram by using an electrocardiograph including three electrocardiogram electrodes and two amplifiers connected to two electrocardiogram electrodes among the three electrocardiogram electrodes.

### BACKGROUND ART

An electrocardiograph (ECG) is a useful device capable of conveniently diagnose a patient's heart condition. The electrocardiograph may be classified into several types depending on the purpose of use. A 12-channel electrocardiogram using 10 wet electrodes is used as a standard electrocardiogram for hospitals to obtain as much information as possible. The user may use the hospital electrocardiograph only when visiting a hospital. An electrocardiogram measurement unit of a patient monitor is used to continuously measure a heart condition of a patient while a small number of wet electrodes are attached to a body of the patient. The patient monitor includes a photoplethysmograph (PPG), and generally, the photoplethysmograph or the electrocardiogram measurement unit includes a function of generating an alarm. A Holter ECG and an event recorder, which a user may use while the user moves, have the following essential features. The features include having a small size, using a battery, and having a storage device for storing measured data and a communication device for transmitting the data. The Holter ECG mainly uses 4 to 7 wet electrodes and cables connected to the electrodes, and provides multi-channel ECG. However, since the Holter ECG has the wet electrodes connected to the cables and attached to the body, the user feels uncomfortable. The electrocardiogram, such as a patch type electrocardiogram, which has recently been disclosed, also requires that electrodes be continuously attached to the body.

Meanwhile, the user may carry the event recorder and instantly measure the ECG when feeling a heart abnormality. Accordingly, the event recorder is compact, is not provided with cables for mainly connecting electrodes, and is provided with dry electrodes on a surface of the event recorder. The event recorder according to the related art is mainly a 1-channel, that is, a 1-lead electrocardiograph for measuring one ECG signal by contacting both hands to two electrodes, respectively.

The electrocardiogram measurement system pursued, that is claimed by the present invention is required to be convenient for individuals to use, required to provide accurate and abundant electrocardiogram measurement values, and required to be small for easy portability. In order to be convenient for personal use the claimed device is required to transmit data through wireless communication with a smartphone or the like. To this end, the claimed device is required to be operated with a battery.

According to the present invention, two limb leads are measured simultaneously and directly in order to provide accurate and abundant electrocardiogram measurement values. As described later, according to the present invention, four leads may be calculated and provided from a measurement value obtained by simultaneously measuring the two limb leads. In general, the terms “channel” and “lead” are used interchangeably in relation to the electrocardiogram. The word “simultaneous” is required to be used very carefully in relation to the electrocardiogram. The word “simultaneous” does not denote “sequential”. In other words, the phrase “to simultaneously measure two leads” is required to literally denote measuring two electrocardiogram voltages at any one moment. Specifically, when lead II is sampled while a voltage of lead I being sampled at a constant sampling period, it can be said that a measurement is simultaneously conducted only when each time point for sampling the lead II is preformed within a time less than the sampling period from every time point for sampling the lead I. In addition, the word “measurement” is also required to be used carefully. The word “measurement” is required to be used only when a physical quantity is actually measured. In digital measurement, one measurement should mean one actual AD conversion. As described later, in an electrocardiogram measurement, when lead I and lead III are measured, for example, lead II may be calculated according to Kirchhoff voltage law. In this case, it is accurate when the lead II is expressed as “calculated”, but the expression “measured” may cause confusion.

One of the most difficult problems in the electrocardiogram measurement is removing power line interference included in an electrocardiogram signal. A driven right leg (DRL) scheme is well-known for removing the power line interference. Actually, most electrocardiograms remove the power line interference by using the DRL scheme. The disadvantage of the DRL scheme is that one DRL electrode is required to be attached to a right foot or a lower right portion of a body. The DRL electrode may be replaced with a ground electrode. Accordingly, in the related art, four electrodes including a DRL electrode are required to be in contact with the body in order to measure two limb leads using the DRL scheme. However, since the DRL electrode is required to be in contact with a right lower abdomen portion, at least one cable and at least one additional electrode should be used or a size of the device is increased. In other words, it is difficult to scale down an electrocardiogram measurement device configured to measure two leads using a DRL electrode to the size of a credit card or a smart watch. Another important issue is that if the DRL electrode is arranged adjacent to another electrode, the voltage of the adjacent electrode is distorted because the voltage of the DRL electrode includes components of an electrocardiogram signal.

It is very difficult to remove power line interference without using the DRL electrode, and a special circuit is required to be used (In-Duk Hwang and John G. Webster, Direct Interference Cancelling for Two-Electrode Biopotential Amplifier, IEEE Transaction on Biomedical Engineering, Vol. 55, No. 11, pp. 2620-2627, 2008). In order to remove the power line interference using a conventional filter, a significantly large quality factor (Q) may be required and it may be difficult to fabricate and calibrate a plurality of filters. is required and it is difficult to fabricate and calibrate a plurality of filters.

Since a dry electrode has a high electrode impedance, the dry electrode causes greater power line interference. However, in the electrocardiogram measurement for user convenience, it may be necessary to use a dry electrode attached to a case surface of the electrocardiogram measurement device without using a wet electrode connected to a cable. In addition, for user convenience, it may be necessary to reduce the number of dry electrodes. It is also required not to bring the DRL electrode into contact with the right leg or a lower right part of a torso. However, in the related art, it is difficult to provide an electrocardiogram measurement device that removes the power line interference while using a minimum number of electrodes, without using a cable.

In order to solve the above problems and requirements, according to the present invention, a cable is not used and a dry electrode is used for the user convenience, and two amplifiers and three electrodes associated with the two limb leads are used to simultaneously measure two limb leads. The electrocardiogram device according to the present invention provides a plate-shaped or watch-shaped electrocardiogram device provided with two dry electrodes separated from each other on one surface and one dry electrode on the other surface, for the user convenience. In addition, the present invention provides a method of removing power line interference without using a DRL electrode.

As described later, the present invention discloses an electrocardiogram measurement unit including three electrodes, in which a power line interference current concentrates and flows through one electrode, two amplifiers connected to the remaining two electrodes other than the one electrode among the three electrodes are used, and the two amplifiers each amplify one electrocardiogram signal to simultaneously measure two electrocardiogram signals. Herein, one amplifier is configured to amplify one signal. One amplifier in an actual configuration may denote an assembly composed of a plurality of amplification stages or active filters cascaded in series.

As described below, the related art has neither presented nor accurately described the technical solution provided by the present invention.

Righter (U.S. Pat. No. 5,191,891, 1993) discloses that a watch-type device is equipped with three electrodes to obtain only one ECG signal.

Amluck (DE 201 19965, 2002) discloses an electrocardiogram having two electrodes on a top surface and one electrode on a bottom surface, but only one lead is measured. In addition, unlike the present invention, Amluck has a display and input/output buttons.

Wei et. al. (U.S. Pat. No. 6,721,591, 2004) discloses that a total of six electrodes including an RL electrode serving as a ground electrode are used. Wei et al. discloses a method of measuring four leads and calculating the remaining 8 leads.

Kazuhiro (JP2007195690, 2007) discloses that a device including a display is provided with 4 electrodes including a ground electrode.

Tso (US Pub. No. 2008/0114221, 2008) discloses a meter including three electrodes. However, in Tso, two electrodes are touched simultaneously with one hand to measure one limb lead, for example, lead I. Since one lead is measured at one time in the above manner, three measurements are required to be sequentially performed to obtain three limb lead rides. In addition, In Tso, an augmented limb lead, which does not need to be directly measured, is also directly measured, and a separate platform is used for the measurement.

Chan et. al. (US Pub. No. 2010/0076331, 2010) discloses a watch including three electrodes. However, Cho et. al. discloses that three leads are measured using three differential amplifiers. Further, in Chan et. al., three filters connected to the amplifiers, respectively, are used to reduce noise of a signal.

Bojovic et. al. (U.S. Pat. No. 7,647,093, 2010) discloses a method of calculating twelve lead signal by measuring three special (non-standard) leads. However, five electrodes including one ground electrode and three amplifiers are provided on both sides of a plate-shaped device to measure three leads including one limb lead (lead I) and two special (non-standard) leads obtained from a chest.

Saldivar (US Pub. No. 2011/0306859, 2011) discloses a cradle of a cellular phone. In Saldivar, three electrodes are provided on one side of the cradle. However, in Saldivar, two of the three electrodes are connected to one differential amplifier 68, and one lead is sequentially measured by using a lead selector (FIG. 4C and paragraph [0054]). In other words, in Saldivar, three leads are sequentially measured one by one.

Berkner et. al. (U.S. Pat. No. 8,903,477, 2014) relates to a method of calculating twelve lead signals through sequential measurements performed while sequentially moving a device by using three or four electrodes placed on both sides of the plate-shaped device. However, a specific measurement method is not presented including an exact internal connection of each electrode. For example, left and right feet have different roles in the ECG measurement. Since Berkner describes that one electrode contacts a foot or a lower limb or torso, it does not distinguish whether the foot is a left foot or a right foot. This ambiguity is also shown in stage 1 of FIG. 6. When three electrodes are used, only one lead may be measured for each when one electrode is placed on the right foot. In addition, Berkner does not present the detailed structure and shape of the claimed device. Most importantly, Berkner uses one amplifier 316 and one filter module 304. When one amplifier 316 and one filter module 304 are used, two measurements must be performed sequentially, for example, to measure two leads. Specifically, Berkner recites “ . . . so in a system comprising only 3 electrodes, the reference electrode is different and shifts for each lead measurement. This may be done by a designated software and/or hardware optionally comprising a switch”. The above technology according to Berkner discloses that one lead is measured at one time by using the one amplifier 316 and the one filter 304. In other words, the method according to Berkner et. al. is not related to the method of the present invention for simultaneously measuring two leads by using three electrodes and two amplifiers.

Amital (US Pub. No. 2014/0163349, 2014) discloses that a common mode cancellation signal is generated from three electrodes in a device provided with four electrodes, and the common mode cancellation signal is coupled to the remaining one electrode to remove the common mode signal (see claim 1). This technique is a traditional DRL method well known before Amital.

Thomson et. al. (US Pub. No. 2015/0018660, 2015) discloses a smartphone case attached with three electrodes. The smartphone case of Thomson has a hole in the front such that the smartphone screen can be seen. However, it fails to present a method for measuring two leads simultaneously using two amplifiers. In addition, since the device of Thomson uses ultrasonic communication, a communication-related issue may be raised when the smartphone and the device are separated by even a slight distance (about 1 foot). In addition, when the user changes a smartphone, the user may not be allowed to use the existing smartphone case according to Thomson.

Drake (US Pub. No. 2016/0135701, 2016) discloses that three electrodes are provided on one side of a plate-shape mobile device to provide 6 leads. However, Drake recites “comprises one or more amplifiers configured to amplify analog signals received from the three electrodes” (paragraph [0025] and claim 4). Therefore, Drake is not clear about a key part of the invention: how many amplifiers are used and how the amplifiers are connected to the three electrodes. In addition, Drake discloses “The ECG device 102 can include a signal processor 116, which can be configured to perform one or more signal processing operations on the signals received from the right arm electrode 108, from the left arm electrode 110, and from the left leg electrode 112” (paragraph [0025]). Therefore, in Drake, three signals are received. In addition, Drake is unclear about whether three signals are received simultaneously or sequentially. In addition, Drake discloses “Various embodiments disclosed herein can relate to a handheld electrocardiographic device for simultaneous acquisition of six leads” (paragraph [0019]), where Drake uses the word “simultaneous” incorrectly, inappropriately and indefinitely. The structure of the device of Drake may be considered to be similar to that of the device of Thomson. In Drake, three electrodes are disposed on one side of the device. Therefore, as with Thomson et al., it is difficult to bring three electrodes into contact with both hands and the body simultaneously.

The device according to Saldivar (WO 2017/066040, 2017) discloses that a lead selection stage 250 is used to connect three electrodes to one amplifier 210. Further, in Saldivar, the device performs six measurements sequentially to obtain six leads. In other words, Saldivar does not simultaneously measure a plurality of leads. In Saldivar, the device also measures three augmented limb leads sequentially and directly.

A photoplethysmograph uses LEDs to emit light to the skin and measures reflected or transmitted light. Recently, the photoplethysmograph accommodated in a smart watch may provide a heart rate, an HRV, and a breathing rate. The HRV provides a lot of information about personal health conditions. The HRV is used for sleep analysis or stress analysis, and also used to detect arrhythmias such as atrial fibrillation. In general, an HRV analysis is performed using ECG. However, recently, it has also been performed using photoplethysmograph. The photoplethysmograph included in a patient monitor measures oxygen saturation and generates an alarm when the oxygen saturation is low. An electrocardiogram measurement unit of the patient monitor generates an alarm when the heart rate calculated using the measured electrocardiogram signal is out of a normal range. When the alarm is generated in the patient monitor, medical staff may take appropriate action on the patient.

From a long time ago, a measurement of blood sugar or electrocardiogram (ECG) has been commercialized as a product. However, a person who wants to measure a plurality of test items including blood sugar and electrocardiogram has the inconvenience of carrying a blood glucose meter and an electrocardiograph separately. Accordingly, there is a need for a device that can measure blood sugar and electrocardiogram with one device. The device capable of measuring blood sugar and electrocardiogram is required to be implemented in a compact size, and small in volume, and mostly powered by batteries, so the device is required to have low power consumption so as to be used for a long time.

A devices capable of measuring blood sugar and electrocardiogram needs a power switch, needs a selection switch to select a blood glucose measurement and an electrocardiogram measurement, and needs a display for indicating measured data. However, the mechanical power switch or selection switch and the display cause problems of increasing a volume or area of the device and consuming battery power, and limitations in miniaturization.

In addition, when a blood glucose measurement circuit and an ECG measurement circuit of the device capable of measuring blood sugar and ECG are separately configured, and the power supply is not separately controlled, all circuits are activated when the power is turned on, thereby causing a problem of increasing the power consumption. Accordingly, it is necessary to operate only the circuits having necessary functions.

### DISCLOSURE

### Technical Problem

Arrhythmia is a terrifying disease that threatens human health and causes an increase in medical expenses. For example, atrial fibrillation, as common as 2% of the population in developing countries, causes blood clots, thereby increasing the risk of stroke. Arrhythmia may be accurately diagnosed when a hospital electrocardiograph is used. However, arrhythmia may not always appear in a patient with arrhythmia and commonly may be intermittent. A Holter electrocardiograph or an event recorder may be used to detect the intermittent arrhythmia. The Holter electrocardiograph is usually used for 1 to 2 days. However, it is very likely that arrhythmia is not found during the period. Meanwhile, the user may carry the event recorder and measure the ECG anytime and anywhere in which symptoms are suspected. However, arrhythmia may be silent or asymptomatic. In this case, the user cannot know when to use the event recorder to measure the ECG.

Recently, methods for diagnosing arrhythmia using a photoplethysmograph built in a wearable device have been reported. Accordingly, when the ECG is measured using the event recorder upon detection of arrhythmia while continuously detecting the expression of arrhythmia using a wearable photoplethysmograph, an accurate arrhythmia diagnosis may be implemented. Albert (David E. Albert, Discordance Monitoring, U.S. Pat. No. 9,839,363 B2, Date of Patent: Dec. 12, 2017) discloses a method and a wearable smart watch for enabling a user to detect arrhythmia using an activity level sensor (e.g., accelerometer) and a photoplethysmograph and then measure one ECG signal using two electrodes. However, Albert does not disclose a method of measuring two electrocardiogram signals using three electrodes.

A photoplethysmograph and an electrocardiograph may be integrated into a single smart watch. However, it may also be necessary that an electrocardiograph is not built into a wearable watch.

The first reason is as follows. Many patients with arrhythmia have diabetes. Accordingly, it may be necessary to fuse an electrocardiograph with a blood glucose meter. However, a separate strip case into which blood test strips are stored, and a needle for obtaining blood by piercing the skin are required to be carried to use the blood glucose meter. Therefore, it is not a significant advantage to mount only a blood glucose meter in a smart watch. More importantly, it is difficult to provide a blood test strip insertion port in a smart watch. In this case, a blood glucose meter and an electrocardiograph may be preferably implemented as a single wireless portable device. And when the photoplethysmograph accommodated in the smart watch generates an arrhythmia occurrence alarm, an electrocardiogram may be measured using the wireless portable device in which the blood glucose meter and the electrocardiograph, which are separate from the smart watch, are accommodated together.

Second, there is an important advantage that even an existing smart watch that includes a photoplethysmograph but does not include an electrocardiograph may be used in the method of the present invention when the software of the photoplethysmograph is updated only.

Third, it is because embedding an electrocardiograph in a smart watch requires a special miniaturization technology and it leads to expensive manufacturing costs. Since a smart watch containing only a photoplethysmograph may be used by young people who are irrelevant to arrhythmia, mass production may be facilitated and manufacturing at low cost may be implemented. For the above three reasons, it is not always necessary to accommodate a photoplethysmograph and an electrocardiograph together in a smart watch. Accordingly, an electrocardiograph may be accommodated in a smart watch or implemented separately.

The present invention has been made in view of the above problems and requirements, and the present invention provides a method of using a photoplethysmograph to detect the occurrence of an arrhythmia, and obtaining two electrocardiogram leads. In addition, the present invention discloses methods in the cases of an electrocardiograph is and is not accommodated in a smart watch accommodated with a photoplethysmograph.

### Technical Solution

The electrocardiogram measurement method using a wearable device according to the present invention, by the device worn on one hand of a user and accommodated therein with a photoplethysmograph, includes: periodically measuring photoplethysmogram; extracting photoplethysmogram parameters by analyzing the measured photoplethysmogram; determining generation of an alarm by using the photoplethysmogram parameters; and generating the alarm based on the determination result, and includes: after the alarm is generated, by an electrocardiograph installed in the wearable device or an electrocardiograph that is separated from the wearable device and portable, receiving electrocardiogram signals through a first electrocardiogram electrode and a second electrocardiogram electrode among three electrocardiogram electrodes coming into contact with a left hand, a right hand, and a left lower abdomen or left leg of a user, respectively; and amplifying two electrocardiogram signals inputted to the first and second electrocardiogram electrodes by using two amplifiers built in the electrocardiograph.

In addition, the electrocardiogram measurement system using a wearable device according to the present invention includes a photoplethysmograph and an electrocardiograph installed in the wearable device or an electrocardiograph which is separated from the wearable device and portable, wherein the photoplethysmograph includes: a photoplethysmogram measurement circuit including at least one LED and at least one photodiode; an AD converter connected to an output terminal of the photoplethysmogram measurement circuit to convert an analog signal into a digital signal; a wireless communication device for transmitting and receiving data; a microcontroller for measuring photoplethysmogram by controlling the photoplethysmogram circuit and the wireless communication device, wherein the microcontroller extracts photoplethysmogram parameters by continuously analyzing the measured photoplethysmogram, determines generation of an alarm by using the extracted photoplethysmogram parameters, and generates the alarm on the basis of the determination result, and the electrocardiograph includes: three dry electrocardiogram measurement electrodes; and two amplifiers for amplifying two electrocardiogram signals induced for two electrocardiogram electrodes out of the three electrocardiogram electrodes.

### Advantageous Effects

The electrocardiograph according to the present invention can provide six electrocardiogram leads simultaneously obtained using the least number of electrodes (specifically, three electrodes) without limitations of time and place due to the convenient portability. The electrocardiogram measurement method according to the present invention can perform electrocardiogram measurements after receiving an alarm from the photoplethysmograph upon the occurrence of intermittent arrhythmia when the user does not recognize symptoms.

### DESCRIPTION OF DRAWINGS

FIG. 1 is a perspective view showing a smart watch having three electrodes according to the present invention.

FIG. 2 is a perspective view showing a portable electrocardiograph having three electrodes according to the present invention.

FIG. 3 is a view showing a method of measuring an electrocardiogram in a six-channel mode using an electrocardiogram measurement device according to the present invention.

FIG. 4 is an electrical equivalent circuit model explaining a principle and an embodiment of removing power line interference in an electrocardiogram measurement device according to the present invention.

FIG. 5 is an electrical equivalent circuit model of the embodiment for simultaneously measuring two channels of an electrocardiogram using two single-ended input amplifiers in the electrocardiogram measurement device according to the present invention.

FIG. 6 is a frequency response of a band pass filter used in the electrocardiogram measurement device according to the present invention.

FIG. 7 is a frequency response of one signal channel in the electrocardiogram measurement device according to the present invention.

FIG. 8 is a block diagram of a circuit built in a smart watch according to the present invention.

FIG. 9 is a flow chart of an arrhythmia alarm generation program according to the present invention.

FIG. 10 is a flow chart showing an electrocardiogram measurement in a smart watch according to the present invention.

FIG. 11 is one embodiment of the electrocardiogram measurement device easily coupled to another object according to the present invention.

### BEST MODE

### Mode for Invention

First, the present invention provides an electrocardiograph including two amplifiers and three electrodes related to the two limb leads to simultaneously measure two limb leads. It is very important in the medical field to measure two limb leads simultaneously. This is because it takes more time and it is inconvenient to measure the two leads sequentially. More importantly, this is because the two limb leads measured at different times may not correlate with each other and may cause confusion in the detailed differentiation of arrhythmia. The present invention provides a method of removing power line interference without using a DRL electrode. The present invention discloses a convenient electrocardiogram measurement method in which two hands are in contact with two electrodes, respectively, and one electrode is in contact with a body, and the electrocardiogram measurement device having a structure suitable for the method.

The appearance, usage, operation principle, and configuration of the electrocardiogram device according to the present invention for the above problems to be solved are as follows. The present invention solves the above problems through systematic circuit design and software implementation.

FIG. 1 shows a smart watch 100 according to the present invention. The smart watch 100 includes three electrodes 111, 112 and 113 provided on a surface of a band. The two electrodes 111 and 112 are installed on an outer surface of the band of the smart watch 100, and one electrode 113 is installed on an inner surface of the band. As shown in FIG. 1, at least one LED 121 and at least one photodiode 122 for measuring photoplethysmogram are provided on a bottom surface of the smart watch, that is, a surface in contact with an arm of the user.

FIG. 2 shows a wireless portable electrocardiograph 200 according to the present invention. The wireless portable electrocardiograph 200 includes three electrodes 211, 212 and 213 provided on a surface thereof. The two electrodes 211 and 212 spaced apart from each other at a predetermined interval are installed on one surface of the wireless portable electrocardiograph 200, and the one electrode 213 is installed on the other surface. A blood test strip insertion port 230 into which a blood test strip 220 may be inserted is provided in the wireless portable electrocardiograph 200 of FIG. 2 according to the present invention to measure blood characteristics such as blood sugar.

According to the present invention, the wireless portable electrocardiograph 200 has been described as an example of measuring electrocardiogram (ECG) and blood sugar. However, it is not limited thereto, and may additionally include a function of measuring blood characteristics other than blood sugar, such as the ketone level or the international normalized ratio (INR) of capillary blood put on the strip. The blood sugar level or ketone level may be measured using an amperometric method. The INR serves as a measure of a blood clotting tendency, and may be measured using an electrical impedance method, an amperometric method, or a mechanical method, for the capillary blood. One blood test strip insertion port 230 into which a blood test strip for the blood characteristic test may be inserted may be provided in a case of the wireless portable electrocardiograph 200 as shown in FIG. 2.

The wireless portable electrocardiograph 200 according to the present invention uses current detectors in order not to use a mechanical power switch or selection switch. The current detectors are always supplied with power required for operation, and awaits to generate an output signal when an event occurs. When the user touches the electrocardiogram electrodes or inserts the blood test strip into the strip insertion port, a loop through which a current may flow is completed when electrically connected to the current detector. Then, the current detector allows a microcurrent to flow through the human body or the blood test strip, and the current detector detects the microcurrent and generates an output signal. When the wireless portable electrocardiograph 200 is not in use, only the current detector operates, the remaining circuits are powered off, and the embedded microcontroller awaits in a sleep mode. When the user generates an event of inserting the blood test strip or touching the electrodes with both hands, the current detector detects a current, and the microcontroller is activated to power on the corresponding circuit.

The method of measuring the electrocardiogram using the smart watch 100 according to the present invention shown in FIG. 1 is similar to the method of measuring the electrocardiogram using the wireless portable electrocardiograph 200 shown in FIG. 2. FIG. 3 shows a method of measuring an electrocardiogram using the wireless portable electrocardiograph 200 according to the present invention, by the user. The user holds the electrode 211 and the electrode 212 provided on one surface of the wireless portable electrocardiograph 200 with both hands, respectively, and brings the electrode 213 provided on the other surface into contact with a left lower abdomen (or left leg) of the user. When the three electrodes are in contact with the human body in the above manner, two limb leads may be measured, and four leads may be additionally calculated and obtained as described below. The measurement method of FIG. 3 is a method provided by the present invention in order to most conveniently obtain a six-channel electrocardiogram. In addition, the present invention provides the most suitable device for the measurement method of FIG. 3.

When the smart watch 100 of FIG. 1 is worn on one hand, one electrode 113 provided on the inner surface of the band comes into contact with the hand. To measure an electrocardiogram, the other hand and the user's left lower abdomen (or left leg) are brought into contact with the two electrodes 111 and 112 provided on the outer surface of the band, respectively.

The principle of the measurement method is as follows. The conventional 12-lead ECG is described, for example, in [ANSI/AAMI/IEC 60601-2-25:2011, Medical electrical equipment-part 2-25:Particular requirements for the basic safety and essential performance of electrocardiographs]. In the conventional 12-lead ECG, three limb leads are defined as follows. Lead I=LA−RA, lead II=LL−RA, and lead III=LL−LA. In the above equations, RA, LA, and LL refer to voltages of the right arm, left arm, and left leg, or torso portions close to the limbs, respectively, where a right leg (DRL) electrode is used in the related art to remove power line interference. One of the three limb leads may be obtained from the other two limb leads based on the above relationship. For example, lead III=lead II−lead I. Three augmented limb leads are defined as follows: aVR=RA−(LA+LL)/2, aVL=LA−(RA+LL)/2, aVF=LL−(RA+LA)/2. Accordingly, the three augmented limb leads may be obtained from two limb leads. For example, it may be obtained from “aVR =−(I+II)/2”. Accordingly, when two limb leads are measured, the remaining four leads may be calculated and obtained. Accordingly, the present invention discloses a device for simultaneously measuring two leads by using three electrodes and two amplifiers to provide six leads. Herein, one amplifier means a configuration amplifying one signal, and one amplifier in an actual configuration may be configured as an assembly composed of a plurality of amplification stages or active filters cascaded in series. A standard 12-lead electrocardiogram includes six leads and six precordial leads (V1 to V6).

Hereinafter, one embodiment of the electrocardiogram measurement device according to the present invention will be described with reference to FIGS. 4 and 5. FIG. 4 is an electrical equivalent circuit model explaining the principle and an embodiment of removing power line interference in the electrocardiogram measurement device according to the present invention. FIG. 5 is an electrical equivalent circuit model of an embodiment for simultaneously measuring two channels of an electrocardiogram by using two single-ended input amplifiers in the electrocardiogram measurement device according to the present invention.

In FIG. 4, a current source 450 is used to model power line interference. Further, in FIG. 4, a human body 430 is modeled as three electrode resistors 431, 432, and 433 connected to each other at one point. In addition, in FIG. 5, one electrocardiogram signal is modeled as one voltage source 461 and 462 present between two electrode resistors. In FIG. 5, since three electrodes are used according to the present invention, two electrocardiogram voltage sources 461 and 462 are modeled as being placed in the human body. This is because there are three electrocardiogram voltages in three electrodes (because the number of cases where two electrodes are selected out of three electrodes is 3), but only two electrocardiogram voltages are independent. The modeling of the power line interference of FIG. 4 and the modeling of the electrocardiogram signal of FIG. 5 are simplified. However, the models are suitable to clarify the problem to be solved. In addition, the above models clearly suggest what to design in the present invention. In addition, when the above models are used, the present invention may be easily understood.

The present invention has been designed based on the above models. Because the related arts did not use the above models, the related arts cannot accurately suggest the solution for the problem.

The present invention may be expressed in various embodiments. However, various embodiments of the present invention are commonly based on the following principle of the present invention. The principle of the present invention has been designed in the present invention for the present invention. The principle of the present invention has a difference in that the DRL electrode is not used compared to the DRL method used in the related art. The problem that has not been solved by the conventional electrocardiogram measurement device that does not use the DRL electrode and that is required to be solved is to remove or reduce power line interference. The power line interference in the electrocardiogram measurement device, is caused by a current source having a substantially infinite output impedance due to a quite high output impedance as shown in FIG. 4, (the power line interference current source is indicated by 450 in FIG. 4). Accordingly, in order to remove the power line interference, the impedance looking into the human body from the power line interference current source is required to be minimized. The impedance looking into the human body from the power line interference current source is the sum of an impedance of the human body and an impedance of the electrocardiogram measurement device. In the end, it is required to minimize the impedance of the electrocardiogram measurement device looking into through the three electrodes. Meanwhile, there exists an impedance called an electrode impedance or electrode resistance between each electrode used to measure the electrocardiogram and the human body (431, 432, and 433 in FIG. 4). Accordingly, in order to minimize the effect of the electrode impedance when measuring an electrocardiogram voltage, the electrocardiogram measurement apparatus should have a high impedance. Accordingly, the electrocardiogram measurement device is required to fulfill two opposing conditions in which a low impedance is necessary to remove the power line interference and a high impedance is necessary to measure the electrocardiogram voltage.

A method that may be considered possible for satisfying the above two opposing conditions is that, for example, in the case of using three electrodes, three resistors having high values are connected to the three electrodes, and the other ends of the three resistors are combined together into one point, and common mode signals of the three electrodes are negatively fed back to the one point where the three resistors are combined. However, it is substantially difficult to use this method. This is because the magnitude of the power line interference current does not decrease due to the large impedance of the power line interference current source. Accordingly, in this case, the power line interference voltage induced across the three resistors is still quite large. Alternatively, the amplifier must be saturated. In addition, since the magnitude of the power line interference current has not decreased and the respective electrode impedances may be different from each other, different power line interference voltages are induced at a high level at each electrode. Therefore, it is difficult to remove the power line interference induced at each electrode even when a differential amplifier is used. This is the difficulty in the conventional art.

Thus, in the present invention, the power line interference current is concentrated and flows through only one of the electrodes installed in the electrocardiogram measurement device. To this end, while three electrodes are connected to the human body, the impedance that the power line interference current source looks into the electrocardiogram measurement apparatus through the one electrode is minimized. Then, the power line interference voltage (indicated by v_{body} 440 in FIG. 4) induced to the human body by the power line interference current source is minimized. Then, since the power line interference voltage induced to the human body is minimized, an input impedance of the other electrodes of the electrocardiogram measurement device can be increased, and the electrocardiogram voltage can be accurately measured. Herein, the important point is that one electrode should not be used for measurement since the power line interference voltage is induced high at the one electrode through which the power line interference current concentrates and flows. Accordingly, in the present invention, when three electrodes are used, two electrodes and two amplifiers to receive electrocardiogram signals from the two electrodes are used for measurement. In particular, it should be noted that a configuration using two differential amplifiers cannot be used in the electrocardiogram measurement apparatus employing three electrodes because only two electrodes should be used for measurement. It should also be noted that, when negative feedback is used, if negative feedback is provided in all frequency bands then the electrocardiogram signals appear at the electrode and mixed with the power line interference voltage, and therefore negative feedback should be provided only at the power line interference frequency. Hereinafter, the present invention will be described in detail with reference to the drawings.

In FIG. 4 and subsequent drawings, the electrocardiogram measurement device 100 of FIG. 4 according to the present invention shows only a part of the device 100 (of FIG. 1) according to the present invention for convenience. In FIG. 4, the electrocardiogram measurement device 100 according to the present invention includes three electrodes 111, 112 and 113, and two amplifier 411 and 412. In FIG. 5, the two amplifiers 411 and 412 used in the present invention are not differential amplifiers but single-ended input amplifiers.

An important feature of the embodiment of the present invention shown in FIG. 4 is that the electrocardiogram measurement device 100 according to the present invention includes an electrode driver 413 represented as a band pass filter. The input of the electrode driver 413 is connected to one electrode 112. The output of the electrode driver 413 drives the electrode 113 through the resistor 423 (it is fed back to the electrode 113). The resonance frequency or peak frequency of the electrode driver, that is, the band pass filter 413, is the same as the frequency of power line interference. In addition, the band pass filter 413 has a high Q. In FIG. 4, the input impedance of the band pass filter 413 is considerably high and the output impedance thereof is considerably low. The element value of the resistor 423 is represented by RO. In the present invention, for simplicity, the resistor 423 is regarded as the output impedance of the electrode driver 413.

According to the present invention, two of the three electrodes are connected to a circuit in the circuit-common through resistors 421 and 422 having a value of Rᵢ. The resistors 421 and 422 may be considered as input impedances of the amplifiers 411 and 412.

In FIG. 4, the reference numeral 430 refers to a model of the human body. A contact resistance, generally referred to as an electrode impedance, exists between the human body and a electrode. In FIG. 4, the electrode impedances (electrode resistances) existing between the human body 430 and the three electrodes 111, 112 and 113 are indicated by the resistances 431, 432, and 433, respectively. Element values of the electrode resistances 431, 432 and 433 are indicated by Rₑ₁,Rₑ₂, Rₑ₃, respectively.

In FIG. 4, the reference numeral 450 refers to a power line interference current source generally used in a power line interference modeling. A current iₙ of the power line interference current source 450 flows in to the circuit-common of the electrocardiogram device 100 according to the present invention through the human body 430 and the three electrodes 111, 112 and 113. When the power line interference currents flowing through the three electrodes 111, 112 and 113 are represented as iₙ₁, iₙ₂, iₙ₃, the following is established based on Kirchhoff's law of current.

iₙ=iₙ₁+iₙ₂+iₙ₃ (Expression 1)

For a circuit analysis, the power line interference induced in the human body 430 is indicated by v_{body}. In FIG. 4, vₙ₁, vₙ₂, vₙ₃ denotes power line interference voltages of electrodes 111, 112 and 113, respectively. In the Expression 1, each current is as follows.

$$\begin{matrix}{i}_{n 1} = \frac{{v}_{body}}{{R}_{i} + {R}_{e 1}} & ( Expression 2 ) \\ {i}_{n 2} = \frac{{v}_{body}}{{R}_{i} + {R}_{e 2}} & ( Expression 3 ) \\ {i}_{n 3} = \frac{{v}_{body} + {v}_{n 2} H ( f )}{{R}_{o} + {R}_{e 3}} & ( Expression 4 ) \\ Herein {v}_{n 2} = \frac{{R}_{i}}{{R}_{i} + {R}_{e 2}} {v}_{body} & ( Expression 5 )\end{matrix}$$

The above −H(f) refers to a transfer function of the band pass filter 413. The following is established using the above Expressions.

$${i}_{n} = \frac{{v}_{body}}{{R}_{i} + {R}_{e 1}} + \frac{{v}_{body}}{{R}_{i} + {R}_{e 2}} + \frac{{v}_{body}}{{R}_{o} + {R}_{e 3}} \frac{{R}_{i}}{{R}_{i} + {R}_{e 2}} H ( f ) + \frac{{v}_{body}}{{R}_{o} + {R}_{e 3}} \qquad ( Expression 6 )$$

According to the present invention, element values of the circuit in FIG. 4 are used to enable the following approximations (Expressions 7 and 8). Expressions 7 and 8 are important componets of the present invention.

Rᵢ>>Rₑ₁, Rₑ₂, or Rₑ₃ (Expression 7)

Rᵢ>>Rₒ (Expression 8)

Then, the following approximation is established.

$${i}_{n} \approx \frac{{v}_{body}}{{R}_{o} + {R}_{e 3}} ( 1 + H ( f ) ) \qquad ( Expression 9 )$$

The following may be obtained from the above

Expression 9.

$${v}_{body} \approx ( {R}_{o} + {R}_{e 3} ) \frac{{i}_{n}}{1 + H ( f )} \qquad ( Expression 10 )$$

In Expression 10, when there is not a feedback, that is, when H(f)=0, the following is established.

v_{body}≈(Rₒ+Rₑ₃)iₙ only when H(f)=0 (Expression 11)

By comparing Equation 10 and Equation 11, it can be seen that the present invention reduces the influence of power line interference current to the amount of feedback, or (1+H(f)) . Therefore, when a size of a gain at the resonant frequency of the band pass filter is |H(fₒ)|>>1, it becomes v_{body}≈0. As described above, the principle of removing the power line interference has been proved in the present invention.

The following can be confirmed using Expressions 2 and 10.

$$\begin{matrix}{v}_{n 1} \approx \frac{{R}_{i}}{{R}_{i} + {R}_{e 1}} ( {R}_{o} + {R}_{e 3} ) \frac{{i}_{n}}{1 + H ( f )} \\ \approx ( {R}_{o} + {R}_{e 3} ) \frac{{i}_{n}}{1 + H ( f )} \\ \approx {v}_{body}\end{matrix} \qquad ( Expression 12 )$$

Now, the following result is obtained for vₙ₂. Based on the above result, v_{body}≈0 and iₙ₃≈iₙ can be used.

Also,

vₙ₃≈v_{body}−iₙ₃Rₑ₃≈−iₙRₑ₃ (Expression 13)

The following may be found based on Expressions 12 and 13.

|vₙ₃|>>|vₙ₁| (Expression 14)

This means that, if is large, as a result of feedback, almost all power line interference current flows through the electrode (the electrode 113 in FIG. 4) to which feedback is provided, and therefore the electrode to which feedback is provided is contaminated by power line interference while the electrodes (the electrodes 111 and 112 in FIG. 4) to which feedback is not provided are hardly influenced by power line interference. This in turn means that only the electrodes to which feedback is not provided should be used for electrocardiogram measurement and the electrode to which feedback is provided should not be used for the measurement. Accordingly, the effect of power line interference cannot be eliminated using a differential amplifier whose input is connected to the electrodes 111 and 113 or a differential amplifier whose input is connected to the electrodes 112 and 113. This is one of the important results of the conventional arts.

Hereinafter, the principle of obtaining two electrocardiogram channel signals by using three electrodes will be described according to the present invention. FIG. is an equivalent circuit when measuring an electrocardiogram by using the electrocardiogram device according to the present invention. In FIG. 5, v₁, v₂, v₃ denotes electrocardiogram signal voltages at the electrodes 111, 112 and 113, respectively. The voltage v₂ at the electrode 112 is obtained as follows by using the principle of superposition.

$${v}_{2} = - {v}_{a} \frac{( {R}_{o} + {R}_{e 3} ) ‖ ( {R}_{i} + {R}_{e 2} )}{( {R}_{i} + {R}_{e 1} ) + ( {R}_{o} + {R}_{e 3} ) ‖ ( {R}_{i} + {R}_{e 2} )} \frac{{R}_{i}}{( {R}_{i} + {R}_{e 2} )} + {v}_{b} \frac{( {R}_{i} + {R}_{e 1} ) ‖ ( {R}_{i} + {R}_{e 2} )}{( {R}_{i} + {R}_{e 1} ) ‖ ( {R}_{i} + {R}_{e 2} ) + ( {R}_{o} + {R}_{e 3} )} \frac{{R}_{i}}{( {R}_{i} + {R}_{e 2} )} - {v}_{2} H ( f ) \frac{( {R}_{i} + {R}_{e 1} ) ‖ ( {R}_{i} + {R}_{e 2} )}{( {R}_{o} + {R}_{e 3} ) + ( {R}_{i} + {R}_{e 1} ) ‖ ( {R}_{i} + {R}_{e 2} )} \frac{{R}_{i}}{( {R}_{i} + {R}_{e 2} )} \qquad ( Expression 15 )$$

In the above Expression 15, the symbol ∥ represents a value of a parallel resistance. In the above Expression 15, the symbol ∥ represents a value of a parallel resistance. As in the previous equations, the conditions of Equations 7 and 8 are assumed. Then, the voltage v₂ is approximated as follows.

$$\begin{matrix}{v}_{2} \approx - {v}_{a} \frac{( {R}_{o} + {R}_{e 3} )}{( {R}_{i} + {R}_{e 1} )} \frac{{R}_{i}}{( {R}_{i} + {R}_{e 2} )} + {v}_{b} - {v}_{2} H ( f ) \\ \approx {v}_{b} - {v}_{2} H ( f )\end{matrix} \qquad ( Expression 16 )$$

Therefore, the voltage v₂ is as follows under the conditions of Equations 7 and 8.

$${v}_{2} \approx {v}_{b} \frac{1}{1 + H ( f )} \qquad ( Expression 17 )$$

It can be seen from the above Expression that v₂≈v_{b} when |H(f)|<<1 in the signal band.

FIG. 6 shows the frequency response of the band pass filter used in the electrocardiogram measurement device according to the present invention. In FIG. 6, a gain at the resonant frequency of the band pass filter is 20 and Q=120. FIG. 7 shows v_{b} may be obtained with an accuracy of 98% at a frequency of 40 Hz or below when the band pass filter of FIG. 6 is used.

Likewise, the voltage v₂ of the electrode 111 is obtained as follows.

$${v}_{1} = + {v}_{a} \frac{{R}_{i}}{( {R}_{i} + {R}_{e 1} ) + ( {R}_{o} + {R}_{e 3} ) ‖ ( {R}_{i} + {R}_{e 2} )} + {v}_{b} \frac{( {R}_{i} + {R}_{e 1} ) ‖ ( {R}_{i} + {R}_{e 2} )}{( {R}_{o} + {R}_{e 3} ) + ( {R}_{i} + {R}_{e 1} ) ‖ ( {R}_{i} + {R}_{e 2} ) + ( {R}_{i} + {R}_{e 1} )} - {v}_{2} H ( f ) \frac{( {R}_{i} + {R}_{e 1} ) ‖ ( {R}_{i} + {R}_{e 2} )}{( {R}_{o} + {R}_{e 3} ) + ( {R}_{i} + {R}_{e 1} ) ‖ ( {R}_{i} + {R}_{e 2} )} \frac{{R}_{i}}{( {R}_{i} + {R}_{e 1} )} \qquad ( Expression 18 )$$

When conditions of Expressions 7 and 8 are used, the voltage v₁ is approximated as follows.

$$\begin{matrix}{v}_{1} \approx + {v}_{a} + {v}_{b} - {v}_{2} H ( f ) \\ \approx {v}_{a} + {v}_{2}\end{matrix} \qquad ( Expression 19 )$$

The above Expression is obtained by using Expression 16. The following Expression 20 is obtained obtained from the above expressions, and vₐ can be obtained by the Expression 20. Based on Expression 20, it can be seen that vₐ may be obtained without the influence of the band pass filter.

v₁−v₂≈+vₐ (Expression 20)

Thus, the principle of obtaining signals of the two electrocardiogram channels by using the two single-ended amplifiers has been described according to the present invention.

Hereinafter, the embodiments according to the present invention will be described with reference to the accompanying drawings. Although the electrocardiogram (ECG) measurement device has been illustrated as including three electrodes in the embodiment, it is not limited thereto, and the electrocardiogram measurement device may be a device including three or more electrodes. Important embodiments of the present invention have already been described using FIGS. 4 to 7 in order to explain the principle of the present invention.

FIG. 8 shows a block diagram of a circuit embedded in the smart watch 100 according to the present invention. In order to clarify the present invention, FIG. 8 does not show all blocks. The smart watch 100 according to the present invention includes a photoplethysmogram measurement circuit 810, and at least one LED 121 and at least one photodiode 122 connected to the photoplethysmogram measurement circuit 810. A power consumption is configured to be small since the duty ratio of a current flowing through the at least one LED 121 is very small. The duty ratio is controlled by a microcontroller 860. The at least one LED 121 radiates light to the skin of the user and the light reflected from the skin of the user is received by the at least one photodiode 122. The reflected light includes photoplethysmogram information. A current flowing through the at least one photodiode 122 is amplified in the photoplethysmogram measurement circuit 810. The amplified signal is converted into a digital signal by an AD converter 850. The digital signal is transferred to the microcontroller 860. The microcontroller 860 analyzes the digital signal by using a pre-built photoplethysmogram analysis program illustrated in FIG. 9. When it is determined that an arrhythmia symptom occurs, an alarm is generated. The alarm may be at least one of sound, light, and vibration.

When the alarm is generated, the microcontroller 860 powers on an electrocardiogram measurement circuit 840. According to the present invention, the three electrocardiogram electrodes 111, 112 and 113 are connected to the electrocardiogram measurement circuit 840 as described above. As described above, the electrocardiogram measurement circuit 840 includes two amplifiers according to the present invention. The electrocardiogram measurement circuit 840 amplifies the two electrocardiogram signals induced at the three electrocardiogram electrodes 111, 112 and 113 through the two amplifiers to generate two outputs. The AD converter 850 receives the two outputs of the electrocardiogram measurement circuit 840, converts the received two outputs into digital signals, and transfers the converted two outputs to the microcontroller 860. The microcontroller 860 may display the outputs of the AD converter 850 onto a display of the smart watch 100. In addition, the microcontroller 860 may transmit the outputs of the AD converter 850 to a smart phone or the like through a wireless communication device 870 and an antenna 880 accommodated in the smart watch 100.

An electrocardiogram measurement process using the portable electrocardiograph 200 of FIG. 2 is as follows. When the user receiving the arrhythmia alarm touches a pair of electrodes 211 and 212 with both hands, the electrocardiogram current detector allows a micro current to flow through the both hands and detects the micro current flowing through the both hands. Then, the current detector generates a signal to switch a mode of the microcontroller accommodated in the portable electrocardiograph 200 from a sleep mode to an activation mode. Then, the microcontroller powers on the electrocardiogram measurement circuit and the AD converter. The electrocardiogram measuring circuit generates two outputs after amplifying two electrocardiogram signals with two amplifiers. The AD converter receives the two outputs of the electrocardiogram measurement circuit, converts the received two outputs into digital signals, and transfers the converted two outputs to the microcontroller. The microcontroller transmits the outputs of the AD converter to a smart phone through a wireless communication device and an antenna accommodated in the portable electrocardiograph 200. After completion of measurement for a predetermined period of time, the microcontroller is switched into the sleep mode and waits for the next touch of both hands.

FIG. 9 shows an operation sequence of the alarm generation program using the photoplethysmogram according to the present invention. The alarm generation program is executed by the microcontroller 860 embedded in the smart watch 100. The photoplethysmograph measures a photoplethysmogram signal (910). The microcontroller 860 embedded in the smart watch 100 performs preprocessing including a process of removing noise included in the measured photoplethysmogram signal (920). By using the preprocessed signal, an HRV extraction 930 of extracting HRV parameters, an HR extraction 932 of extracting HR parameters, and a BR extraction 934 of extracting BR parameters are performed. In order to extract the HR parameters, the photoplethysmogram signal is first order differentiated or second order differentiated, a position of a peak value is called R, and a time between R and the next R (R-R interval) is obtained first. There are various ways to obtain the HRV parameters. For the HRV a standard deviation of the R-R interval in the time domain may be used . The BR parameters may be obtained by extracting low frequency components of the photoplethysmogram. In the HRV determination 940, it is determined as arrhythmia when the HRV increases or decreases beyond a predetermined set value. In the HR and BR determination 942, it is determined as arrhythmia when the HR increases beyond a predetermined set value without an increase in the BR. When it is determined as arrhythmia in the HRV determination 940 or when it is determined as arrhythmia in the HR and BR determination 942, an alarm is generated (950).

FIG. 10 is a flowchart illustrating an operation of the electrocardiograph accommodated in the smart watch 100 according to the present invention when electrocardiogram is measured. When the alarm is generated in the photoplethysmograph (1010), the microcontroller 860 powers on the electrocardiogram measurement circuit 840 (1020). This may be performed by connecting an output pin of the microcontroller 860 to the electrocardiogram measurement circuit 840 and setting a voltage of the output pin as high. Next, the current detector 830 is used to check whether the pair of electrodes 111 and 112 are in contact with both hands (1030). When the both hands are in contact, the microcontroller 860 starts measuring the electrocardiogram (1040). The microcontroller 860 performs an AD conversion in accordance with a preset AD conversion period and obtains an AD conversion result. According to the present invention, two electrocardiogram signals are measured. Data on the measured electrocardiogram may be transmitted to the smartphone (1050) and stored in a memory accommodated in the smart watch 100 (1060). After a preset measurement time, such as 30 seconds, elapses, the microcontroller 860 sets a voltage of the output pin of the electrocardiogram measurement circuit 840 as low to power off the electrocardiogram measurement circuit 840 (1070) and the electrocardiogram measurement is terminated.

It is very important that the present invention includes the step 1020 in which the microcontroller 860 of FIG. 10 powers on the electrocardiogram measurement circuit 840 and the step 1070 in which the microcontroller 860 powers off the electrocardiogram measurement circuit 840. This is because that the power consumption of the photoplethysmograph and the electrocardiograph is required to be saved or reduced as much as possible since the photoplethysmograph and the electrocardiograph used in the present invention operate by a battery. According to the present invention, the photoplethysmograph is required to operate continuously, but the electrocardiograph is turned on only when measuring the electrocardiogram and turned off when not measuring the electrocardiogram to reduce the power consumption of the battery.

The present invention has been described with respect to the smart watch 100 of FIG. 1. However, the present invention may be implemented in various forms in addition to the smart watch 100 of FIG. 1. In other words, the electrocardiograph for measuring six limb leads using the three electrocardiogram electrodes may have a ring shape and may have a shape of using a clip easily attached to pants. In addition, even when it is implemented in the form of a smart watch, the electrode 113 of FIG. 1 may be installed on the bottom surface of the smart watch 100, that is, at a position adjacent to a position where the at least one LED 121 and the at least one photodiode 122 are installed. According to the present invention, when the photoplethysmograph and the electrocardiograph are implemented in one device including the ring shape or the clip shape easily attached to the pants, at least one electrode (the electrode 113 in the above description) may be preferably installed in a position adjacent to a position where the at least one LED and the at least one photodiode are installed.

The electrocardiogram measurement device according to the present invention may be implemented in a form easily coupled to another object so as to be always worn. FIG. 11 shows an example of the wearable device according to the present invention capable of immediately measuring an electrocardiogram when intended to measure the electrocardiogram after attached to the pants. FIG. 11 shows that two clips 111 and 112 serving as two electrodes are used to attach an electrocardiogram measurement device 1100 according to the present invention to an inner side of the pants, that is, between the pants and the body of the user. When the electrocardiogram measurement device 1100 is attached to a left lower abdomen portion of the pants by using the clip 111 and the clip 112 upon use, the electrode 113 and the photoplethysmograph 1110 automatically come into contact with a left lower abdomen portion of the user. When the photoplethysmograph 1110 sends an alarm or an electrocardiogram is necessary to be measured, the user contacts a finger of a left hand to the clip 111 and a finger of a right hand to the clip 112.

The device in FIG. 11 may implement only the electrocardiograph without the photoplethysmograph, so that the electrocardiogram when arrhythmia occurs may be measured according to the present invention. In this case, when an alarm is generated from the photoplethysmograph of the smart watch in which the photoplethysmograph is implemented, the user touches the two clips. Then, the electrocardiogram current detector of the electrocardiograph detects a current flowing between the both hands to power on the electrocardiogram measurement circuit. When the electrocardiogram measurement is finished, the electrocardiogram measurement circuit may be powered off.

As described above, the electrocardiogram measurement method and system according to the present invention have been described in detail, however, the present invention is not limited thereto. The present invention may be modified in various forms that meet the intent of the present invention.

### INDUSTRIAL APPLICABILITY

The electrocardiograph accommodated in the smart watch or a portable electrocardiograph carried separately from the smart watch according to the present invention may be convenient to carry, may be easily used regardless of time and place, and may allow electrocardiogram information of multiple channels to be obtained. Particularly, even when asymptomatic arrhythmia occurs, the user receiving an arrhythmia alarm measures the electrocardiogram, so that an accurate diagnosis may be obtained later.

1. An electrocardiogram measurement method using a wearable device, the electrocardiogram measurement method comprising:
by the a wearable device in which a photoplethysmograph coming into contact with a skin of a user is accommodated, measuring a photoplethysmogram;
extracting photoplethysmogram parameters by analyzing the measured photoplethysmogram;
determining generation of an alarm by using the photoplethysmogram parameters; and generating the alarm based on the determination result, and comprising:
after the alarm is generated, by an electrocardiograph installed in the wearable device or an electrocardiograph that is separated from the wearable device and portable, powering on an electrocardiogram measurement circuit;
receiving electrocardiogram signals through a first electrocardiogram electrode and a second electrocardiogram electrode among at least three electrocardiogram electrodes coming into contact with a left hand, a right hand, and a left lower abdomen or left leg of a user, respectively;
amplifying two electrocardiogram signals inputted to the first and second electrocardiogram electrodes by using two amplifiers built in the electrocardiograph;
powering off the electrocardiogram measurement circuit; and calculating six limb leads by using the two electrocardiogram signals.

2. The electrocardiogram measurement method using the alarm of the photoplethysmograph of claim 1, wherein the photoplethysmogram parameters include a heart rate, a heart rate variability, and a breathing rate.

3. The electrocardiogram measurement method using the alarm of the photoplethysmograph of claim 1, wherein the determination of the alarm generation includes an arrhythmia occurrence.

4. The electrocardiogram measurement method using the alarm of the photoplethysmograph of claim 3, wherein the determination of the arrhythmia generation includes an increase of a heart rate without an increase of a breathing rate.

5. The electrocardiogram measurement method using the alarm of the photoplethysmograph of claim 3, wherein the determination of the arrhythmia generation includes an increase or decrease of a heart rate variability.

6. The electrocardiogram measurement method using the alarm of the photoplethysmograph of claim 3, wherein the determination of the arrhythmia generation is performed by deep learning.

7. The electrocardiogram measurement method using the alarm of the photoplethysmograph of claim 1, wherein the two amplifiers are single-ended input amplifiers.

8. The electrocardiogram measurement method using the alarm of the photoplethysmograph of claim 1, wherein six limb lead signals of lead I, lead II, lead III, lead aVR, lead aVL, and lead aVF are obtained using the measured two electrocardiogram signals.

9. The electrocardiogram measurement method using the alarm of the photoplethysmograph of claim 1, wherein the wireless portable electrocardiograph includes a blood characteristic measurement unit that measures one or more of a blood sugar level, a ketone level, or an INR.

10. An electrocardiogram measurement system using a wearable device, the electrocardiogram measurement system comprising:
a photoplethysmograph, and an electrocardiograph installed in the wearable device or an electrocardiograph that is separated from the wearable device and portable, wherein the photoplethysmograph includes:
a photoplethysmogram measurement circuit including at least one LED and at least one photodiode;
an AD converter connected to an output terminal of the photoplethysmogram measurement circuit to convert an analog signal into a digital signal;
a wireless communication device for transmitting and receiving data; and
a microcontroller for measuring photoplethysmogram by controlling the photoplethysmogram measurement circuit and the wireless communication device, wherein the microcontroller extracts photoplethysmogram parameters by continuously analyzing the measured photoplethysmogram, determines generation of an alarm by using the extracted photoplethysmogram parameters, and generates the alarm on the basis of the determination result, and the electrocardiograph includes:
at least three dry electrocardiogram measurement electrodes; and
two amplifiers for amplifying two electrocardiogram signals induced at two electrocardiogram electrodes out of the at least three electrocardiogram electrodes.

11. The electrocardiogram measurement system using the alarm of the photoplethysmograph of claim 10, wherein the photoplethysmogram parameters include a heart rate, a heart rate variability, and a breathing rate.

12. The electrocardiogram measurement system using the alarm of the photoplethysmograph of claim 10, wherein the determination of the alarm generation includes an arrhythmia occurrence.

13. The electrocardiogram measurement system using the alarm of the photoplethysmograph of claim 12, wherein the determination of the arrhythmia generation includes an increase of a heart rate without an increase of a breathing rate.

14. The electrocardiogram measurement system using the alarm of the photoplethysmograph of claim 12, wherein the determination of the arrhythmia generation includes an increase or decrease of a heart rate variability.

15. The electrocardiogram measurement system using the alarm of the photoplethysmograph of claim 12, wherein the determination of the arrhythmia generation is performed by deep learning.

16. The electrocardiogram measurement system using the alarm of the photoplethysmograph of claim 10, wherein the two amplifiers are single-ended input amplifiers.

17. The electrocardiogram measurement system using the alarm of the photoplethysmograph of claim 10, wherein signals of six channels of lead I, lead II, lead III, lead aVR, lead aVL, and lead aVF are obtained using the measured two electrocardiogram signals.

18. The electrocardiogram measurement system using the alarm of the photoplethysmograph of claim 10, wherein the wireless portable electrocardiograph includes a blood characteristic measurement unit that measures one or more of a blood sugar level, a ketone level, or an INR.
